(19) United States
(12) Patent Application Publication
(10) Pub. No.: US 2005/0277996 A1
(43) Pub. Date: Dec. 15, 2005

(54) Method of reducing pressure in a nucleus pulposus

(76) Inventor: Ronald J. Podhajsky, Boulder, CO (US)

Correspondence Address: UNITED STATES SURGICAL,;A DIVISION OF TYCO HEALTHCARE GROUP LP, 150 GLOVER AVENUE, NORWALK, CT, 06856, US

(21) Appl. No.: 11/141,176

(22) Filed: May 31, 2005

Related U.S. Application Data
(60) Provisional application No. 60/575,218, filed on May 28, 2004.

(51) Int. Cl. A61N 1/18

(52) U.S. Cl. 607/46

(57) ABSTRACT

A method for reducing intervertebral pressure is provided. The method includes providing an electrode and a generator. The electrode includes a proximal end and a distal end. The generator is operatively connected to the proximal end of the electrode and is configured to supply radiofrequency current thereto. The method also includes inserting at least a portion of the distal end of the electrode into the nucleus pulposus of an intervertebral disc and activating the generator to heat the nucleus pulposus. The intervertebral disc has a first lateral side and a second lateral side. The nucleus pulposus has a major axis and a minor axis. The electrode may be inserted into the intervertebral disc through its first lateral side and/or its second lateral side and may be substantially parallel to the major or minor axis of the nucleus pulposus.

### CROSS REFERENCE TO RELATED APPLICATION

The present application is related to and claims priority to U.S. Provisional Application Ser. No. 60/575,218 filed May 28, 2004, the entire contents of which are hereby incorporated by reference.

### BACKGROUND

1. Technical Field

The present disclosure relates generally to advances in medical systems and procedures for prolonging and improving human life. More particularly, this disclosure relates to a method for heating and/or applying external pressure to the intervertebral disc to reduce the internal pressure of the intervertebral disc.

2. Background of Related Art

The use of radiofrequency electrodes for ablation of tissue in the body or for the treatment of pain is known. In a typical application, a radiofrequency probe or a resistive heating probe may be constructed in an elongated, cylindrical configuration and inserted into the body to a target tissue which is to be treated or ablated. In the case of a radiofrequency probe, there may be an exposed conductive tip portion and an insulated portion of the probe. When connected to an external source of radiofrequency power, heating of tissue occurs near the exposed conductive portion of the probe, whereby therapeutic changes in the target tissue near the conductive tip are created by the elevation of temperature of the tissue. Thermal probes can also be made by resistive heating of a portion of the probe so as to heat surrounding tissue by thermal conduction. By reference, the products of Radionics, Inc., located in Burlington, Mass., include commercially available radiofrequency generators and electrode systems of varied configurations. A paper by Cosman, et al, entitled “Theoretical Aspects of Radiofrequency Lesions in the Dorsal Root Entry Zone”, Neurosurgery, December 1984, Vol. 15, No. 6, pp. 945-950, describes aspects of tissue heating using radiofrequency electrodes and probes.

The use of thermal therapy in and around the spinal column is also known. Heating of an intervertebral disc to relieve pain is described in commonly-assigned U.S. Pat. No. 5,433,739 entitled “Method and Apparatus for Heating an Intervertebral Disc for Relief of Back Pain” and in commonly-assigned U.S. Pat. No. 5,571,147 entitled “Thermal Dennervation of an Intervertebral Disc for Relief of Back Pain,” the contents of each patent being incorporated herein by reference. In these patents, electrodes are described for either radiofrequency or resistive thermal heating of all or a portion of the intervertebral disc. Straight, curved, and flexible-tipped electrodes are described for this purpose.

It may be desirable to treat the posterior or posterior/lateral portion of the intervertebral disc for the indication of mechanical degeneration of the disc and discogenic back pain. Pain can be derived from degeneration or compression of the intervertebral disc in its posterior or posterior/lateral portions. There is some innervation of the intervertebral disc near the surface of the disc and also within its outer portion known as the annulus fibrosis. Mechanical damage such as fissures or cracks within the disc caused by age or mechanical trauma may result in disc innervation which is believed to be associated with painful symptoms. The existence of relatively high pressure within the disc may also be associated with painful symptoms.

Accordingly, the present disclosure is directed to a method which provides a reduction of the pressure within the intervertebral disc. The method includes heating the nucleus pulposus with an RF electrode and/or applying pressure to the external portion of the intervertebral disc.

### SUMMARY

The present disclosure relates to a method for reducing intervertebral pressure. The method includes providing an electrode and a generator. The electrode includes a proximal end and a distal end. The generator is operatively connected to the proximal end of the electrode and is configured to supply radiofrequency current. The method also includes inserting at least a portion of the distal end of the electrode into the nucleus pulposus of an intervertebral disc and activating the generator to heat the nucleus pulposus via the electrode. The intervertebral disc has a first lateral side and a second lateral side. The nucleus pulposus has a major axis and a minor axis.

In one embodiment, the range of the portion of the electrode inserted into the nucleus pulposus is from about 1 mm to about 20 mm, and may be equal to 10 mm.

In one embodiment, the electrode may be inserted into the first lateral side of the intervertebral disc. It is envisioned for the electrode to be inserted substantially parallel to the major axis of the nucleus pulposus or substantially parallel to the minor axis of the nucleus pulposus. The electrode may also be removed from the first lateral side and re-inserted into the second lateral side of the intervertebral disc. These steps would allow for two portions of the intervertebral disc to be heated.

It is envisioned for external pressure to be applied to the intervertebral disc. The amount of external pressure applied may be in the range of about 5 psi to about 100 psi, and may be equal to about 40 psi.

In one embodiment, the heat supplied by the generator may be in the range of about 70° C. to about 110° C., and may be equal to about 90° C. The generator may be activated for an amount of time in the range of about 50 seconds to about 200 seconds, and may be equal to about 120 seconds.

It is also envisioned for the intervertebral disc to be in the range of about 1 year-old to about 3 years-old.

Additionally, the step of inserting an introducer into the intervertebral disc to help position the electrode may be included in the present method.

### BRIEF DESCRIPTION OF THE DRAWINGS

Embodiments of the present disclosure are described hereinbelow with reference to the drawings wherein:

FIG. 1 is a schematic illustration of an intervertebral disc;

FIG. 2 is a schematic illustration of the intervertebral disc of FIG. 1 with an introducer and an electrode inserted therein through a first lateral side of the disc and substantially parallel to its major axis;

FIG. 3 is a schematic illustration of the intervertebral disc of FIG. 1 with the introducer and the electrode inserted therein through a second lateral side of the disc and substantially parallel to its major axis;

FIG. 4 is a schematic illustration of the intervertebral disc of FIG. 1 with the introducer and the electrode inserted therein substantially parallel to the disc's minor axis; and

FIG. 5 is a flow chart illustrating steps of the method of the present disclosure.

### DETAILED DESCRIPTION

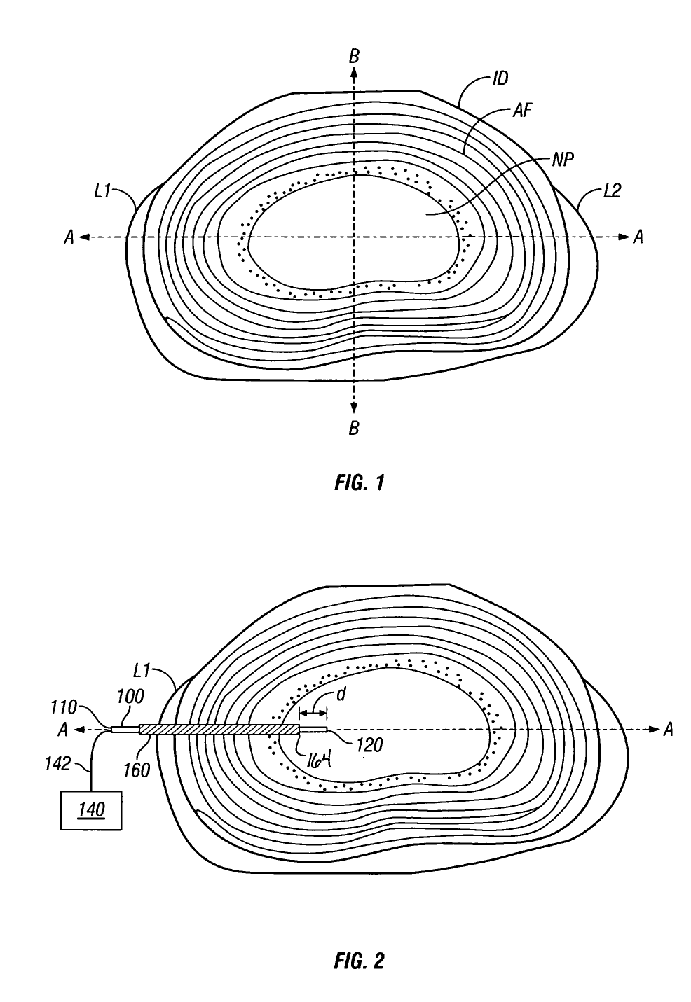

Referring initially to FIG. 1, an intervertebral disc is shown and indicated by reference letters ID. A nucleus pulposus NP and an annulus fibrosus AF are shown within the intervertebral disc ID. A major axis of the intervertebral disc ID is indicated by dashed line A-A and a minor axis of the intervertebral disc is indicated by dashed line B-B. A first lateral side L1 and a second lateral side L2 of the intervertebral disc ID are also illustrated.

Referring now to FIG. 2, an electrode 100 is shown inserted into the nucleus pulposus NP of the intervertebral disc ID. Electrode 100 includes a proximal end 110 and a distal end 120. The electrode 100 is shown inserted through an introducer 160, which facilitates the entry of the electrode 100 into the intervertebral disc ID. The proximal end 110 of the electrode 100 is connected to a generator 140 via cable 142. The generator 140 is designed to supply radiofrequency current to the electrode 100. The distal end 120 of the electrode 100 extends a distance “d” past a distal end 164 of the introducer 160. This distance d may range from about 1 mm to about 20 mm and may be approximately equal to 10 mm.

The introducer 160 and the electrode 100 of FIG. 2 are shown inserted through the first lateral side L1 of the intervertebral disc ID and substantially parallel to the major axis A-A of the intervertebral disc ID.

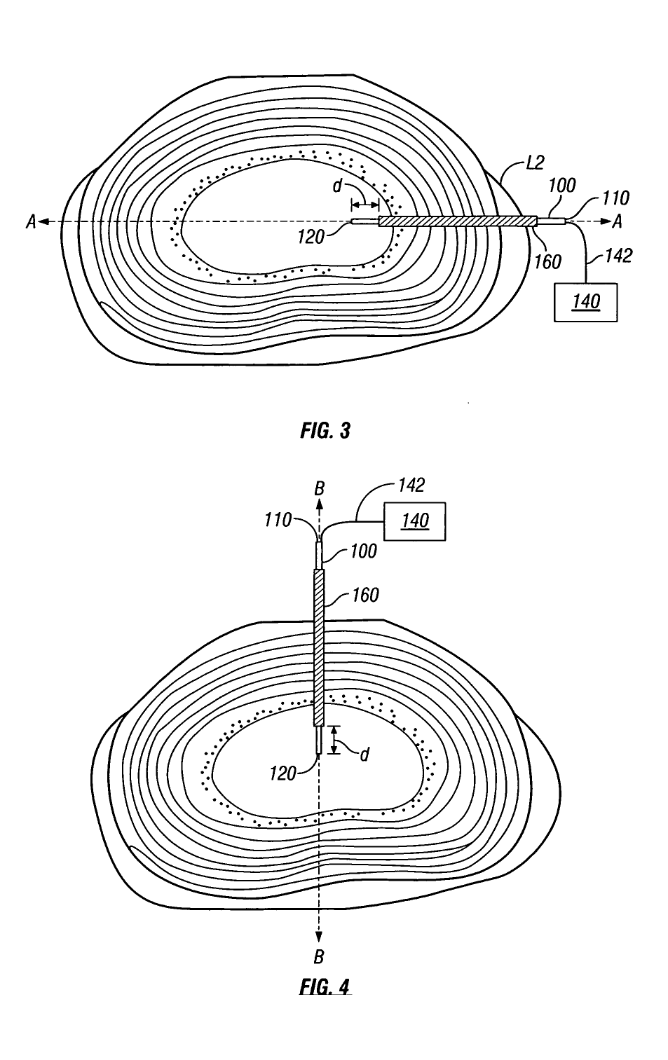

With reference to FIG. 3, the introducer 160 and the electrode 100 are shown inserted through the second lateral side L2 of the intervertebral disc ID and substantially parallel to the major axis A-A of the intervertebral disc ID. As seen in FIG. 4, the introducer 160 and the electrode 100, are shown inserted into the intervertebral disc ID substantially parallel to the minor axis B-B of the intervertebral disc ID.

It is envisioned that the electrode 100 may be inserted into the nucleus pulposus NP without an introducer 160. It is further envisioned that the electrode 100 and/or introducer 160 may be inserted into the intervertebral disc ID from the side opposite the side shown in FIG. 4 (substantially parallel to the minor axis B-B). It is also envisioned for more than one electrode 100 and/or introducer 160 to be in the intervertebral disc ID at the same time. For example, the electrode 100 and introducer 160 of FIG. 2 may remain in the intervertebral disc ID while a second electrode 100 and introducer 160 of FIG. 3 is inserted. In such an embodiment, there would be two electrodes 100 in the nucleus pulposus NP—one being inserted from the first lateral side L1 of the intervertebral disc ID and the other being inserted from its second later side L2.

It is envisioned for the generator 140 to supply heat in the range of about 70° C. to about 110° C., and may be approximately 90° C. It is further envisioned for the generator 140 to be activated for a period of time in the range of about 50 seconds to about 200 seconds, and may be approximately equal to 120 seconds.

It is also envisioned for the intervertebral disc ID to be “young,” in the range of about 1 year-old to about 3 years-old.

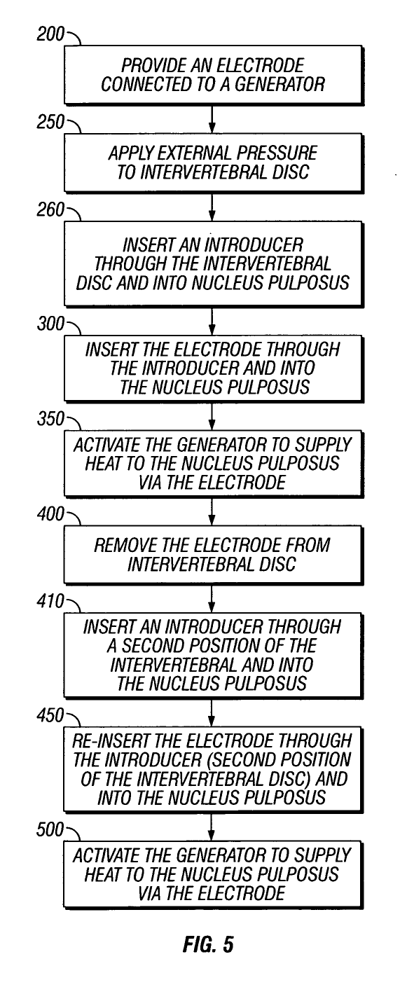

The method of the present disclosure is illustrated by the flow chart of FIG. 5. A first step of the method, indicated by reference numeral 200, is to provide an electrode 100 connected to a generator 140 via cable 142.

A second (optional) step 250 is to apply external pressure to the intervertebral disc ID. The amount of pressure may be in the range of about 5 psi to about 100 psi, and may be equal to approximately 40 psi. Pressure is applied to intervertebral disc ID using any method know by one having skill in the art, such as, for example, mechanical pressure, fluid pressure, and the like.

A third (optional) step 260 is to insert the introducer 160 through the intervertebral disc ID and into the nucleus pulposus NP.

A fourth step 300 is to insert the electrode 100 into the nucleus pulposus NP. Desirably, the electrode 100 may be inserted into the nucleus pulposus NP through the introducer 160, however, it is envisioned that the electrode 100 may be inserted into the nucleus pulposus NP without an introducer 160.

A fifth step 350 is to activate the generator 140 to supply heat to the nucleus pulposus NP via the electrode 100.

Other optional steps may also be included. Such optional steps include: a sixth step 400—removing the electrode 100 from the intervertebral disc ID; a seventh step 410—inserting the introducer 160 (or a second introducer) through a second position in the intervertebral disc ID (other than the position in step 260) and into the nucleus pulposus NP; an eighth step 450—inserting the electrode 100 (or a second electrode) through the introducer 160 (or the second introducer) through the second position in the intervertebral disc ID and into the nucleus pulposus NP; and a ninth step 500—activate the generator 140 to supply heat to the nucleus pulposus NP via the electrode 100.

There have been described and illustrated herein several embodiments of a method for reducing the pressure in a nucleus pulposus. While particular embodiments of the disclosure have been described, it is not intended that the disclosure be limited thereto, as it is intended that the disclosure be as broad in scope as the art will allow and that the specification be read likewise. Therefore, the above description should not be construed as limiting, but merely as exemplifications of various embodiments. Those skilled in the art will envision other modifications within the scope and spirit of the claims appended hereto. For example, it is envisioned that the generator 140 and the electrode 100 are dimensioned and configured to be connectable with each other and are not operatively connected to each other. It is also envisioned that the electrode 100 is inserted into and/or heats the annulus fibrosus AF.

1. A method for reducing intervertebral pressure including the steps of:
providing an electrode and a generator, the electrode including a proximal end and a distal end, the generator being operatively connected to the proximal end of the electrode and being configured to supply radiofrequency current to the electrode;
inserting at least a portion of the distal end of the electrode into the nucleus pulposus of an intervertebral disc, the intervertebral disc having a first lateral side and a second lateral side, the nucleus pulposus having a major axis and a minor axis; and
activating the generator to supply an amount of heat to the nucleus pulposus for a period of time.

2. The method of reducing intervertebral pressure according to claim 1, wherein the portion of the electrode inserted into the nucleus pulposus has a length in the range of about 1 mm to about 20 mm.

3. The method of reducing intervertebral pressure according to claim 2, wherein the portion of the electrode inserted into the intervertebral disc has a length approximately equal to 10 mm.

4. The method of reducing intervertebral pressure according to claim 1, wherein the electrode is inserted into the first lateral side of the intervertebral disc.

5. The method of reducing intervertebral pressure according to claim 4, further including the steps of:
removing the electrode from the first lateral side of the intervertebral disc; and
inserting the electrode into the second lateral side of the intervertebral disc.

6. The method of reducing intervertebral pressure according to claim 1, further including the step of:
applying external pressure to the intervertebral disc.

7. The method of reducing intervertebral pressure according to claim 6, wherein the amount of external pressure applied to the intervertebral disc is in the range of about 5 psi to about 100 psi.

8. The method of reducing intervertebral pressure according to claim 7, wherein the amount of external pressure applied to the intervertebral disc is approximately 40 psi.

9. The method of reducing intervertebral pressure according to claim 1, wherein the intervertebral disc is in the range of about 1 year-old to about 3 years-old.

10. The method of reducing intervertebral pressure according to claim 1, wherein the electrode is inserted substantially parallel to the major axis of the nucleus pulposus.

11. The method of reducing intervertebral pressure according to claim 1, wherein the electrode is inserted substantially parallel to the minor axis of the nucleus pulposus.

12. The method of reducing intervertebral pressure according to claim 1, wherein the amount of heat supplied by the generator is in the range of about 70° C. to about 110° C.

13. The method of reducing intervertebral pressure according to claim 12, wherein the amount of heat supplied by the generator is approximately equal to 90° C.

14. The method of reducing intervertebral pressure according to claim 1, wherein the amount of time the generator is activated is in the range of about 50 seconds to about 200 seconds.

15. The method of reducing intervertebral pressure according to claim 14, wherein the amount of time the generator is activated is approximately equal to 120 seconds.

16. The method of reducing intervertebral pressure according to claim 1, further including the step of inserting an introducer into the intervertebral disc, wherein the inserting an introducer step being after the providing step and before the inserting the electrode step.

17. The method of reducing intervertebral pressure according to claim 16, wherein at least a portion of the distal end of the electrode is inserted into the nucleus pulposus through the introducer.
